(19) United States
(12) Patent Application Publication
(10) Pub. No.: US 2004/0138159 A1
(43) Pub. Date: Jul. 15, 2004

(54) Oligonucletides for regulating the gene coding for tnf$g(a) and/or genes controlled thereby and use thereof

(76) Inventors: Jacques Pourel, Vandoeuvre Les Nancy (FR); Jean-Yves Jouzeau, Chaligny (FR); Isabelle Chary-Valckenaere, Nancy (FR); Amr Abid, Jarville La Malgrange (FR); Horea Porumb, Bobigny (FR); Eliane Taillandier, Paris (FR); Patrick Netter, Nancy (FR)

Correspondence Address: BURNS DOANE SWECKER & MATHIS L L P, POST OFFICE BOX 1404, ALEXANDRIA, VA, 22313-1404, US

(21) Appl. No.: 10/471,716

(22) PCT Filed: Mar. 15, 2002
(86) PCT No.: PCT/FR02/00939

(30) Foreign Application Priority Data
Mar. 16, 2001 (FR) 01/03619

(51) Int. Cl. A61K 48/00; C07H 21/04

(52) U.S. Cl. 514/44; 536/23.2

(57) ABSTRACT

The invention is directed toward oligonucleotides capable of directly or indirectly modulating the expression of the gene encoding tumor necrosis factor alpha (TNFα) in various species and/or of genes under its control. These oligonucleotides are characterized in that they comprise:

a constant portion-GGGGNGGG-, where N=T, A or U

in the 5′ to 3′ or 3′ to 5′ orientation,

complementary to a target sequence,

two variable portions,

located on either side of the constant portion, conferring on the oligonucleotides a specificity of direct or indirect interaction with the target in the TNFα gene of the species considered

in which the base at each of the ends, adjacent to the constant portion, is a purine.

Applications in diagnosis and in therapeutics, and as research tools.

The invention relates to oligonucleotides capable of regulating the gene encoding tumor necrosis factor alpha (abbreviated to TNFα) and/or genes under its control, and to applications thereof in diagnosis, and in therapeutics and as research tools.

TNFα is a pro-inflammatory cytokine which modulates the growth, differentiation and function of a large number of cell types, hence a broad spectrum of biological activities. It is involved in the physiopathology of many conditions, in particular chronic inflammatory diseases, such as rheumatoid arthritis. Specifically, TNFα plays a central role in the synovial inflammation and the osteocartilaginous destruction which characterizes this disease in which the perpetuation of the inflammation is associated with an excess production of pro-inflammatory cytokines such as interleukin 1β (IL1β), interleukin 6 (IL6) and interleukin 8 (IL8).

TNFα also plays a central role in immune and tumor-related pathological conditions, and its action on the central nervous systems causes fever, anorexia and weight loss.

Specific inhibition of TNFα therefore constitutes one of the major aims in terms of therapeutics and for understanding the physiopathology of these conditions.

A demonstration thereof has been provided by the beneficial clinical effects obtained by administering anti-TNFα chimeric monoclonal antibodies (1) or soluble TNFα receptors to individuals suffering from rheumatoid arthritis.

It has also been shown, through experimental studies in rats, that neutralizing or inhibiting the production of TNFα leads to a decrease in the production of IL1βand, consequently, those of IL6 and IL8 (2).

However, the practical advantage of using anti-TNFα antibodies or soluble receptors appears to be limited in the long-term treatment of rheumatoid arthritis.

Another approach consists in inhibiting the expression of the TNFα gene using oligonucleotides with a view to gene therapies.

The development of antisense oligonucleotides capable of reducing the translation of TNFα has thus been reported. In this regard, various studies have shown the effectiveness and the selectivity of antisense oligonucleotides directed against the mRNAs of cytokines, of growth factors, of adhesion molecules, and of enzymes for biosynthesis of inflammation mediators (3) and (4). The use of triple helix-forming oligonucleotides has also been shown to be effective, in vitro in inhibiting the expression of genes, such as those encoding the human oncogenes c-myc, c-erbB, Ha-ras and HER-2/neu.

Anti-gene oligonucleotides, making it possible to decrease the transcription of the gene encoding TNFα, have also been described (5).

In other studies, oligonucleotides have been used to induce cleavage of the target nucleic acid (ribozyme strategy), or to act as a competitive ligand for a protein with respect to its natural nucleic acid target (decoy strategy). By forming a three-dimensional structure with itself, an oligonucleotide can also specifically recognize a protein via an aptameric effect.

The inventor's research in this field has related to the development of oligonucleotides capable of inhibiting not only the gene encoding TNFα in mammals, including in humans, but also genes under its control.

Their studies have thus shown that, by designing oligonucleotides of given sequence, it is possible to achieve such results at very low doses. Thus, on cells in culture, effective doses of such oligonucleotides are within a concentration range of nanomolar order, whereas the doses reported in the prior art for blocking TNFα production are of micromolar order.

The invention is therefore directed toward novel oligonucleotides for inhibiting the transcription of the TNFα gene and of genes under its control.

The aim of the invention is also the uses of these oligonucleotides for such inhibitions, in diagnosis, research and therapeutics.

The oligonucleotides of the invention are capable of directly or indirectly modulating the expression of the gene encoding tumor necrosis factor alpha (TNFα) in various species and/or of genes under its control. These oligonucleotides are characterized in that they comprise:

a constant portion-GGGGNGGG-, where N=T, A or U

in the 5′ to 3′ or 3′ to 5′ orientation,

complementary to a target sequence,

two variable portions,

located on either side of the constant portion, conferring on the oligonucleotides a specificity of direct or indirect interaction with the target in the TNFα gene of the species considered,

in which the base at each of the ends, adjacent to the constant portion, is a purine.

These oligonucleotides are more particularly characterized in that they comprise from 8 to 25 nucleotides, preferably of the order of 20 nucleotides.

The target for these oligonucleotides consists of a deoxyribonucleic or ribonucleic acid sequence, or a protein which interacts directly or indirectly with this sequence.

It is, for example, a target located on the coding or noncoding strand of the DNA or of the RNA of eukaryotic and prokaryotic living organisms and viruses.

In the case of the nuclear or extra nuclear DNA of eukaryotes, the target sequence can be intronic or exonic or can overlap the two types of DNA.

As a variant, the target consists of a double helix of nucleic acid comprising at least the sequence:

5′-CCCCGCCC-3′,
or
3′-GGGGCGGG-5′.

If the interactions between the oligonucleotides and the duplex target involve hydrogen bonds, the latter can be of the perfect or imperfect Watson-Crick type, of the Hoogsteen or reversed Hoogsteen type, or other types. They are possibly formed with one of the strands or both strands of the duplex.

This target, in the case of the human gene encoding TNFα, is located in the promoter, upstream of the transcription initiation codon and of the TATA box, and at position 563-570 of the nucleotide sequence (accession number X02910 and X02159 of Gene Bank) published by Nedwin et. al., (6). Given the role of the TATA box in the transcriptional mechanism and the distance between the target and this box, the interaction of an oligonucleotide with this target can then bring about a reduction in transcription. Specifically, this interaction of the oligonucleotides with the target can inhibit the attachment of a regulatory protein which is essential for the expression of the target gene (transcription factor for example). This interaction can also introduce irreversible damage (cleavages cross-links) into the DNA molecule, making it locally unable to undergo gene expression.

In the other mammals, and still in the case of the gene encoding TNFα, this target is also found in the promoter upstream of the TATA box. Thus, in the Muridae (for example in Mus musculus BALB/c), this target is at position 934-941 of the nucleotide sequence (accession number U68415 of Gene Bank) published by Iraqi and Teale (7). In the Bovidae (for example in Bos taurus), the target is at position 1131-1138 of the sequence having the accession number AF011926 from Gene Bank. In the Hominidae (for example in Pan troglodytes), the target is at position 344-351 of the sequence with the accession number U42626 from Gene Bank.

It is possible for the oligonucleotides of the present invention not to interact directly with the target consisting of the nucleic acid, but with one of the proteins involved in the recognition of this target and/or the regulation of transcription of the TNFα gene.

The constant portion is complementary to the target sequence, i.e. a guanine of the sequence of the oligonucleotide corresponds to a cytosine of the target sequence, and a guanine of the reverse sequence of the oligonucleotide corresponds to a cytosine in the sequence of the target. However, the cytosine, complementary to the guanine, is replaced in the sequence of the oligonucleotide with another pyrimidine base, thymine, or with a purine base, adenine or uracil. It will be noted that two orientations are possible.

The sequence of the two variable portions will be determined by the sequence of the gene the expression of which it is desired to modulate.

To modulate the expression of the gene encoding TNFα in the Hominidae, for example in humans or chimpanzees, the oligonucleotides comprise variable portions of sequences AAGAAA and AGGAGAG, located, respectively, on the 5′ side and on the 3′ side in the 5′γ3′ direction or on the 3′ side and on the 5′ side in the 3′φ5′ direction.

To modulate the expression of the gene encoding TNFα in the Muridae, for example in rats or mice, the oligonucleotides comprise variable portions of sequences TCGAAAA and AGAAGG, located, respectively, on the 5′ side and on the 3′ side in the 5′γ3′ direction, or on the 3′ side and on the 5′ side in the 3′φ5′ direction. In the Bovidae, the variable portions correspond to the sequences CGGAAA and AGAAGTG, located, respectively, on the 5′ side and on the 3′ side in the 5′γ3′ direction, or on the 3′ and on the 5′ side in the 3′φ5′ direction.

The oligonucleotides thus designed can also interact not with the TNFα gene, but with transcription factors. In fact, the sequence AAGAAAGGGNGGGGGAGAG is, for example, recognized by the transcription factors GKLF (Gut-enriched Krueppel Like-Factor), MZF1 (Mycloid Zinc Finger protein 1) or Sp1 (Stimulating protein 1).

Choosing the two variable portions such that they are complementary on at least 4 nucleotides will give oligonucleotides which exhibit a “stem-loop” structure. In this case, the oligonucleotides comprise variable portions of sequences NNNNNNNCCCCAA and GGGGNNNNN, located, respectively, on the 5′ side and on the 3′ side in the 5′γ3′ direction, or on the 3′ side and on the 5′ side in the 3′φ5′ direction, of the constant portion. N may be T, A, C, G or U. The oligonucleotides thus designed will interact with the target proteins by structural recognition.

Advantageously, the oligonucleotides of the invention comprise from 8 to 30 nucleotides, preferably of the order of 20 nucleotides, in particular 21 nucleotides.

The oligonucleotides of the invention may comprise modifications of their phosphodiester chains and/or of additional reactive groups located at their ends.

The aim of these modifications introduced into the oligonucleotides is to increase the resistance of these molecules to nucleolytic degradation, and/or to promote their interactions with their targets, and/or to allow degradation/modification reactions specific for the DNA targets, and/or to increase their intracellular penetration, and/or to improve the crossing of biological membranes.

The following examples of modifications or of substitutions are given by way of illustration and do not constitute a limitation to the present invention.

The modifications or substitutions include N-alkylphosphoramidates, phosphorothioates, phosphotriesters, methylphosphonates, short alkyl chains, hetero atoms, and cycloalkyl or heterocyclic inter-sugar bridges. The phosphodiester bonds of the oligonucleotide backbone can be replaced with 3′γ5′ phosphoramidate bonds or with bonds, polyamides; the nucleic acid bases are then linked directly or indirectly to the aza nitrogen of the polyamide. The modifications or substitutions can also occur in the 2′-position of the carbohydrate components of the oligonucleotides, comprising one of the following substitutions: OH, SH, SCH₃, F, OCN, OCH₃OCH₃, OCH₃O(CH₂)ₙCH₃, O(CH₂)ₙNH₂, O(CH₂)ₙCH₃, Cl, Br, CN, CF₃, OCF₃, O-alkyl, S-alkyl, N-alkyl, O-alkenyl, S-alkenyl, N-alkenyl, SOCH₃, SO₂CH₃, ONO₂, NO₂, N₃, NH₂, heterocycloalkyl, heterocycloalkaryl, aminoalkylamino, polyalkylamino, substituted silyl, a cleavable RNA group, a reporter group, a group which improves the pharmacokinetic properties of the oligonucleotides, a group which improves the pharmacodynamic properties of the oligonucleotides, or other substitutions having the same properties. The modifications also include those of the type 2′-methoxyethoxy (2′-O—CH₂CH₂OCH₃), 2′-methoxy (2′-O—CH₃) or 2′-propoxy (2′-O—CH₂CH₂CH₃).

All the modifications in the 2′-position of the nucleic acid sugars stated above can take place in the 3′-position of the sugars, in the position 3′ of the terminal nucleotide or 5′ of the terminal nucleotide. The oligonucleotides can also have mimetic sugars, such as cyclobutyls instead of pentofuranosyls, xylose-derived sugars, sugars in blocked conformation, and isomers in the D- or L- form. The substitution or the modification of the nucleic acid bases with universal bases (such as hypoxanthine) can also take place. The nucleotides may also be of α-anomerism instead of β-anomerism as in natural nucleic acids.

Lipophilic portions can be grafted onto the oligonucleotides of the present invention. The aim of these substitutions is to modify the charge and/or the hydrophilicity of the oligonucleotides in order to increase the crossing of biological membranes. These lipophilic portions include, for example, cholesterol, a cholesteryl group, cholic acid, thioethers (hexyl-S-tritylthiol, for example), thiocholesterol, aliphatic chains (dodecanediol or undecyl residues, for example), phospholipids (dihexadecyl-rac-glycero-3-H-phosophonate, for example), polyamine chains or polyethylene glycol, adamantane acetic acid, a palmityl group, an octadecylamine group or a hexylamino carbonyloxycholesterol group.

Groups can be substituted in order to facilitate detection of the oligonucleotides. Peptides can be covalently bonded to the oligonucleotides of the present invention in order to assist the recognition of organs, of tissues or of cell types. All these modifications or substitutions can of course be combined with one another. They can concern all the nucleotides or only some of them. The oligonucleotides of the present invention may be part of a DNA or of an RNA which is circular or circularizable.

The study of the oligonucleotides of the invention has made it possible to demonstrate their effectiveness and their specificity for modulating the expression of genes encoding products exerting pathogenic effects in humans and animals, more particularly of the gene encoding TNFα, or of genes under its control.

The invention is therefore directed toward pharmaceutical compositions, characterized in that they contain a therapeutically effective amount of at least one oligonucleotide as defined above, in combination with a pharmaceutically acceptable vehicle.

These compositions can be used in a curative or prophylactic capacity.

The treatment of a patient needing this type of therapy is based on the administration of a pharmaceutical form of the oligonucleotide at doses of between 0.01 μg and 100 g per kg of weight of the patient. The choice of the dose will depend on the age of the patient, on the severity of the affliction and on the nature of the pathology. The frequency of administration may be form one or more times a day, to once every twenty years. After the treatment, the patient is monitored in order to evaluate the improvements which have occurred in his or her pathological condition and the symptoms. Given these observations, the doses may then be increased when the patient does not respond significantly to the treatment, or decreased in the opposite case.

In certain cases, it is preferable to treat the patient with the oligonucleotides of the present invention in combination with conventional, already existing therapies, in order to increase the effectiveness of the treatment.

After the treatment has been successful, it is sometimes necessary to administer maintenance doses to the patient, in order to avoid reappearance of the symptoms, and a relapse. In this case, the doses administered will be between 0.01 μg and 100 g per kg of weight of the patient, at frequencies which can range from one or more times a day up to once every 20 years.

The routes of administration of the pharmaceutical preparation (or formula) of the oligonucleotides of the present invention will vary depending on whether a local or systemic treatment is desired. Administrations will therefore be carried out transmucosally (ophthalmic route, vaginal route, rectal route, intranasal route, pulmonary route, sublingual route) orally, cutaneously or parenterally. The parenteral administrations can be epidural, intravenous (direct or by infusion), subcutaneous, intraperitoneal, intrathecal, intracardiac, intramuscular or intra-articular administrations.

The pharmaceutical formulation and the conditioning which the oligonucleotides will have to undergo in order to constitute medicinal products will depend on the routes of administration. In the case of transmucosal and cutaneous administrations, for example, the oligonucleotides can be administered in the form of lotions, of creams, or ointment, of drops, of suppositories, of liquids or of powders or as an aerosol. The oligonucleotides can also be administered using active (mechanical or electrical) or passive devices. The excipients will be components which have no pharmacological action but which are required for the manufacture, the administration or the conservation of the oligonucleotides of the present invention.

In the case of oral administration, the oligonucleotides of the present invention can be administered, for example, in the form of a powder, of a granule, of a capsule, of a cachet, of a tablet, of a gelatin capsule, or a lozenge, or a suspension in aqueous or nonaqueous solution. The parenteral administrations can be given in the form of a sterile aqueous solution which may contain buffers or other additives. Active (mechanical or electrical) devices can also be used.

The dosage of the pharmaceutical preparations will depend on the severity and on the response to treatment of the pathological condition to be treated. The optimal doses to be administered to the patients will depend on the pharmacological activity of the oligonucleotides. This activity is in general determined in vitro or in vivo on experimental animal models by the 50% effective doses (ED₅₀)

The inflammatory model of experimental arthritis in rats, using complete Freund's adjuvant (CFA), is a model which is widely used to evaluate the pharmacological activity of anti-rheumatic medicinal products. The pharmacological activity of the oligonucleotides of the present invention can thus be evaluated in this model. The parameters which will be taken into account to determine this pharmacological activity will be:

fever;

edema in the back paws;

locomotor functions;

integrity of the cartilage, by analyzing the biosynthesis of proteoglycans and the degradation of the latter in the patellar cartilage;

production of TNFα systemically and locally.

The invention is also directed toward the use of the oligonucleotides defined above, as tools for research and for diagnosis.

It thus uses a method of diagnosis in which the oligonucleotides of the present invention, which may or may not be included in a kit, are used to make an account of the accumulation, associated with a pathological process, of an active transcriptional factor. Physiological processes are closely linked to the transcriptional activity of a large number of genes. As regards this “physiological” gene transcription, it is dependant on the balance between the active and inactive state of a restricted number of transcriptional factors. A pathological process is often associated with an increase or a decrease in the number of active transcriptional protein factors. The oligonucleotides of the present invention, in solution or on a fixed support, single-stranded or double-stranded, coupled or not coupled to a tracer such as a fluorophore, a chromophore or an enzyme, can be used to quantify an active transcriptional factor. It has thus been reported that an oligonucleotide in solution coupled to a fluorophore, called “Molecular Beacons”, by interacting with a protein, produces a fluorescence which is readily quantifiable by common methods of spectrofluorometry (16). It has also been reported that a double-stranded oligonucleotide attached to a solid support can be used to quantify the protein-nucleic acid interaction during high-throughput screening (17).

In research and in development, the oligonucleotides of the present invention, which may or may not be included in a kit, are used to study physiopathological mechanisms at the cellular and/or molecular level. They can thus be used to evaluate the impact of a certain number of molecules on these same physiopathological mechanisms.

The kits containing the oligonucleotides defined above and the reagents required to carry out research and diagnosis also fall within the field of protection of the invention.

In these kits, the oligonucleotides are in solution or on a support, single-stranded or double-stranded, coupled or not coupled to a tracer, such as a fluorophore, a chromophore or an enzyme.

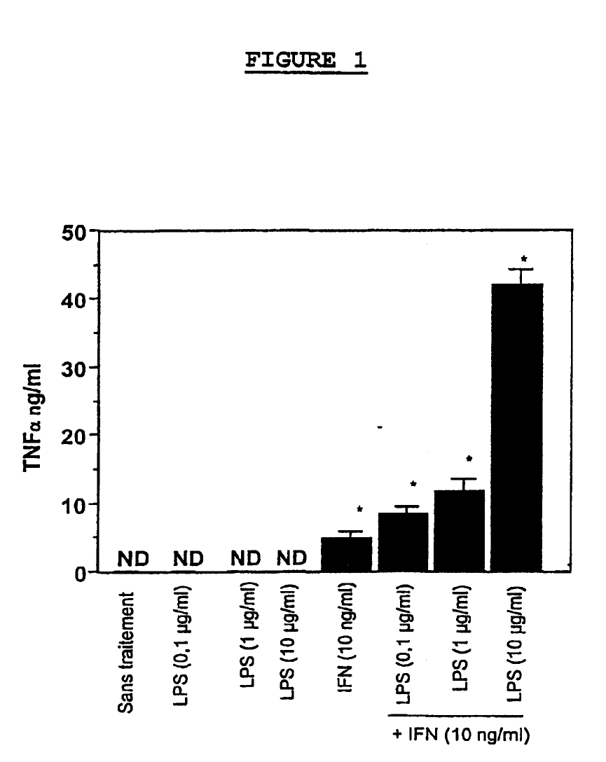

Other characteristics and advantages of the invention are given in the following examples with reference to FIGS. 1 to 5, which represent, respectively:

FIG. 1: The effect of gamma-interferon (IFN) and of lipopolysaccharide (LPS) on the production of TNFα in THP-1 cells. The results represent the mean of three independent assays ± the standard deviation. ND, not detectable; *, significantly different from the cells without treatment (p<0.01);

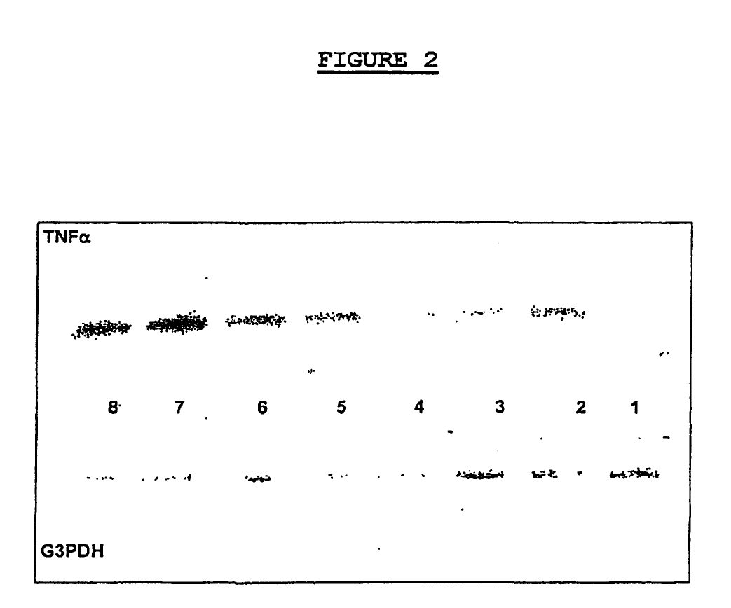

FIG. 2: The effect of gamma-interferon (IFN) and of lipopolysaccharide (LPS) on the synthesis of TNFα mRNAs in THP-1 cells. 1, treated with 0.1% (v/v) of DMSO; 2, 0.1 μg/ml LPS; 3, 1 μg/ml LPS; 4, 10 μg/ml LPS, 5, 10 ng/ml IFN; 6, IFN (10 ng/ml)+LPS (0.1 μg/ml); 7, IFN (10 ng/ml)+LPS (1 μg/ml); 8, IFN (10 ng/ml)+LPS (10 μg/ml);

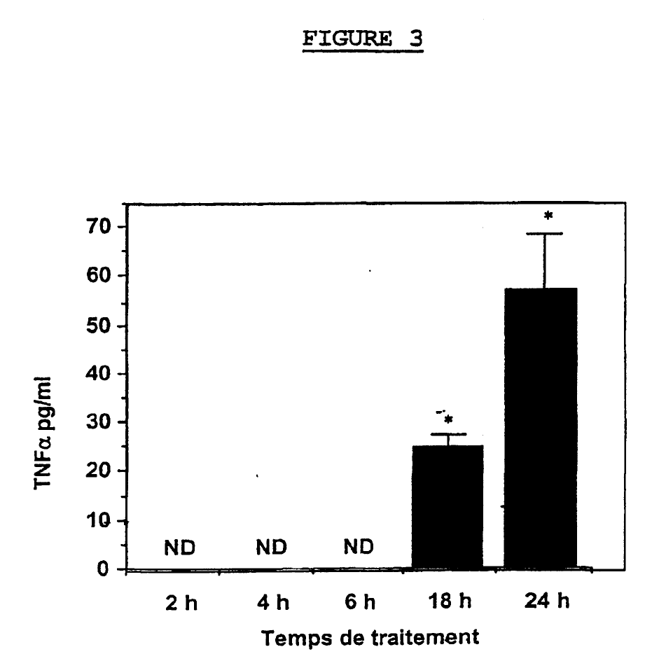

FIG. 3: Kinetics of TNFα production in stimulated THP-1 cells. The cells were treated with IFN (10 ng/ml)+LPS (10 μg/ml). The results represent the mean of three independent assays ±the standard deviation. ND, not detectable; *, significantly different from the 2 h cells (p<0.01);

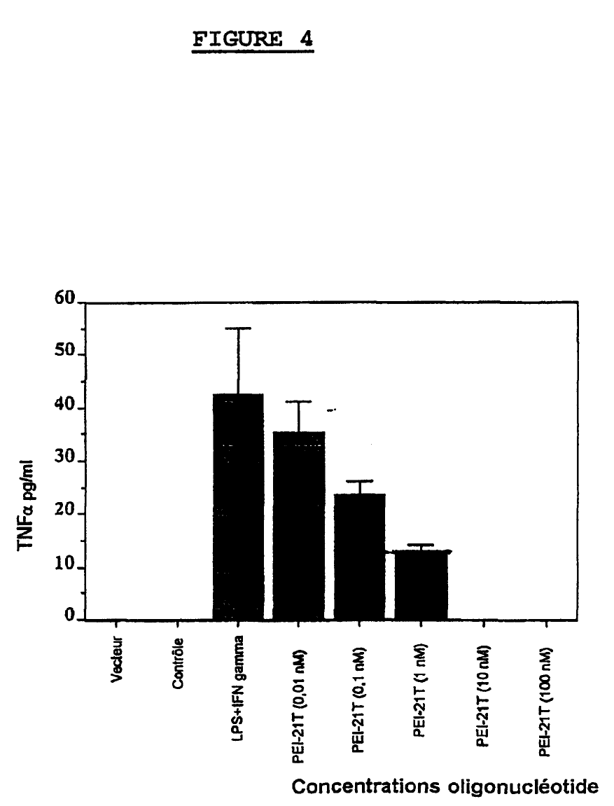

FIG. 4: The effect of an oligonucleotide according to the invention on the production of TNFa in THP-1 cells stimulated with the combination LPS-IFN (10 μg/ml-10 ng/ml). It is the oligonucleotide 21T, which comprises a constant portion in the 5′ to 3′ orientation, containing N=T; and two variable portions AAGAAA and AGGAGAG, located, respectively, on the 5′ side and on the 3′ side in the 5′γ3′ direction. The results represent the mean of three independent assays ±the standard deviation; *, significantly different from the control cells (p<0.0001); **, significantly different from the LPS—IFN cells (p<0.005); ND, not detectable;

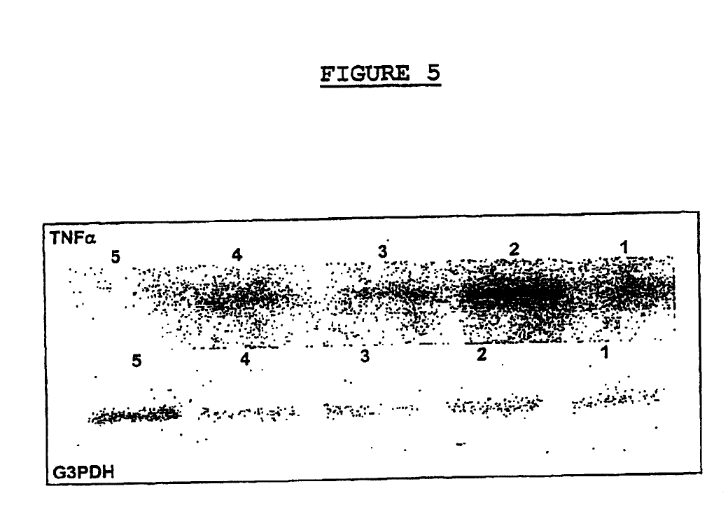

FIG. 5: The effect of an oligonucleotide 21T on the synthesis of TNFα mRNAs in THP-1 cells stimulated with the combination LPS-IFN (10 μg/ml-10 ng/ml). 1, cells treated with 0.1% (v/v) DMSO; 2, cells stimulated with LPS-IFN; 3, stimulated cells treated with 1 nM; 4, stimulated cells treated with 10 nM; 5, stimulated cells treated with 100 nM.

### OLIGONUCLEOTIDE SYNTHESIS

The oligonucleotides are synthesized chemically according to conventional methods and using equipment and devices which exist on the market.

Treatment of Human Cells

The results reported in the examples were obtained with a human monocyte cell line since monocytes constitute the major source of TNFα in the inflammatory reaction. It is the THP-1 cell line originating from a 1-year-old child suffering from acute monocytic leukemia. This line has a monocytic morphology and differentiates into macrophages (8). The lipopolysaccharide and gamma-interferon, which constitute potent cytokine inducers, were used as inducer molecules.

The THP-1 human monocyte line (ECACC, Cerdic, France) is cultured in RPMI 1640 medium, supplemented with 2 mM L-glutamine, 100 IU/ml penicillin, 100 μg/ml streptomycin, 0.25 μg/ml of fungizone, 10% (v/v) of fetal calf serum decomplemented at 56° C. for 30 minutes (all the products come from Life Technologies, France).

The cultures are maintained in an incubator at 37° C. in a humid atmosphere containing 5% CO₂ For the various experiments, the cells are placed in culture in 24-well plates (Costar, France) at a density of 10⁶ cells/well.

The production of TNFα is induced by treating the THP-1 cells with a mixture of blood lipopolysaccharide (LPS) of Escherichia coli 055:B5 (Sigma, France) and human gamma-interferon (IFN; Tebu, France). The action in synergy of these two factors induces overexpression of the TNFα gene in the THP-1 cells (9).

The cells are deposited in a volume of 0.5 ml/well of complete culture medium and incubated for 18 hours at 37° C., and then 0.5 ml of complete culture medium is added, which medium contains:

either various concentrations (0.1-10 μg/ml) of LPS (the LPS is dissolved in DMSO; final concentration 0.1% v/v);

or 10 ng/ml of IFN (dissolved in the medium);

or 10 ng/ml of IFN in combination with various concentrations (0.1-10 μg/ml) of LPS.

After incubation for 18 hours, the samples are removed and centrifuged at 12 000 g for 3 minutes. The supernatants and also the pellets are stored separately at −80° C. until their respective uses for determining the TNFα contents and for preparing the RNAs.

The TNFα concentrations are determined by an ELISA (Enzyme Immuno Sorbent Assay; Biotrak, Amersham, France) method of the sandwich type. The assay was used according to the supplier's instructions, using human TNFα as standard.

The total RNAs are separated from the cell pellets by the method of Chomczynski et al., (10) using the Trizol® solution (Life Technologies, France). The TNFα mRNA synthesis is analyzed by the method of reverse transcription, followed by a polymerase chain amplification.

The sequence of the primer used for the reverse transcription is as follows:

RDT₁₇: 5′-GACTCGAGTCGACAAGCTTTTTTTTTTTTTTTTT-3′

The procedure for obtaining the cDNA strand is as follows (11):

| | |
|---|---|
| Total RNA | 1 μg |
| Primer (20 μM) | 1 μl |
| Ultra-pure sterile water | q.s. 20 μl |

The mixture is heated for 10 minutes at 70° C., and then rapidly cooled in crushed ice. The following solutions (Life Technologies, France) are successively added to this mixture:

| | |
|---|---|
| Reverse transcription buffer | 10 μl |
| dNTP (10 mM) | 2.5 μl |
| DTT (100 mM) | 5 μl |
| RNase inhibitor | 1 μl |
| SuperScript Rnase H- reverse transcriptase | 1 μl |
| Ultra-pure sterile water | q.s. 50 μl |

The reverse transcription reaction takes place at 42° C. for 2 hours. Once the reaction has finished, the mixture is diluted to 1/10 in ultra-pure sterile water, and then stored at −80° C.

The amplification reaction takes place in the following reaction mixture:

| | |
|---|---|
| Reaction buffer | 5 μl |
| dNTP (10 mM) | 1 μl |
| 3′ primer (20 μM) | 1 μl |
| 5′ primer (20 μM) | 1 μl |
| MgCl₂ (50 mM) | 1.5 μl |
| RT solution (cDNA) | 5 μl |
| Taq DNA polymerase (5 U/μl) | 0.5 μl |
| Ultra-pure sterile water | q.s. 50 μl |

The amplification reaction comprises three steps (11).

Synthesis of the Second cDNA Strand:

| | |
|---|---|
| 94° C. (denaturation of the double strand) | 1 minute |
| 60° C. (primer hybridization) | 1 minute |
| 72° C. (elongation) | 40 minutes |

Amplification of the Double-Stranded cDNA (30 Cycles):

| | |
|---|---|
| 94° C. | 1 minute |
| 60° C. (primer hybridization) | 1 minute |
| 72° C. (elongation) | 1 minute |

Final Extension:

| | |
|---|---|
| 72° C. | 10 minutes |

The primers for the TNFα were designed on the basis of the published mRNA sequence (12). The sequences of the two primers used are as follows:

| | |
|---|---|
| TNF-F: | 5′GGCCCAGGCAGTCAGATCATCT-3′ |
| TNF-R; | 5′TAGACCTGCCCAGACTCGGCAA-3′ |

The design of the primers for glyceraldehyde 3-phosphodehydrogenase (G3PDH), used as standard, was determined on the basis of the published mRNA sequence (13). The sequences of the primers used are as follows:

| | |
|---|---|
| HG3PDH-F: | 5′-TATTGGGCGCCTGGTCACCAGG-3′ |
| HG3PDH-R: | 5′-CTGGAGGAGTGGGTGTCGCTGT-3′ |

The amplification gives products which are 933 bp in size for G3PDH, and 462 bp in size for TNFα. The specificity of the amplification was verified by the size of the products obtained and by sequencing the cDNAs.

The amplification products are separated on a 1% (w/v) agarose gel under a constant voltage. The gel contains ethidium bromide (0.2 μg/ml) (Life Technologies, France). Once the migration is finished, the gel is photographed under UV (by transillumination) using a CCD camera.

FIG. 1 gives the results of stimulation of the production of TNFα. The various concentrations of LPS have no effect on the production of TNFα in the THP-1 cells. On the other hand, the addition of 10 ng/ml of IFN to the three concentrations of LPS leads to a dose-dependent increase in the production of TNFα. The IFN on its own also causes an increase in the production of TNFα. On the other hand, this increase remains significantly less than that produced by the combination IFN-LPS.

The effect of the various concentrations of LPS combined or not combined with IFN, on the expression of the TNFα gene, was also studied using a semi-quantitative method, reverse transcription followed by chain amplification.

The expression of the gene encoding G3PDH was used as standard to which the increases in TNFα expression were related. The expression of the gene encoding G3PDH is relatively insensitive to treatments with LPS or with cytokines (14).

As shown in FIG. 2, treatment of the THP-1 cells with LPS causes a slight increase in the expression of the TNFα gene. Treatment with IFN on its own also causes an increase in the TNFα mRNAs. The combination of IFN and LPS leads to a dose-dependant increase in the mRNAs. In addition, this increase is greater than that caused by the treatment with LPS or IFN alone.

Other work related to the study of the kinetics of TNFα production by the stimulated THP-1 cells. FIG. 3 shows that treatment of the THP-1 cells with the combination IFN-LPS (10 ng/ml IFN and 10 μg/ml LPS) causes a significant time-dependant increase with respect to the production of TNFα.

Inhibition of TNFα Production

The THP-1 cells are transfected using ExGen 500 (Euromedex, France), which is a vector consisting of a combination of ethyleneimine polymers (PEI) (15).

As described above, one million cells are deposited per well in a volume of 0.5 ml of complete culture medium, and incubated for 18 hours at 37° C. and under 5% CO₂, and then 0.1 ml of transfection solution is added to the cells, and the cells are then incubated for 4 hours at 37° C. under 5% CO₂. At the end of this period, 0.4 ml of stimulating solution (10 ng/ml of IFN+10 μg/ml of LPS per well in complete culture medium) is added to the cells, and said cells are then incubated for 18 hours at 37° C. under 5% CO₂. After incubation for 18 hours, the samples are taken and centrifuged at 12 000 g for 3 minutes. The supernatants and also the pellets are stored separately at −80° C. until their respective uses for determining the TNFα content and preparing the RNAs.

The transfection solution consists of the oligonucleotides diluted to the desired concentrations in 0.05 ml of a 0.9% (w/v) NaCl solution. In addition, 0.01 ml of ExGen 500 is diluted in 0.05 ml of the same NaCl solution. The 0.05 ml of vector solution are added to the 0.05 ml of the oligonucleotide solution. The mixture is vortexed, briefly centrifuged, and then incubated for 15 minutes at ambient temperature (time required for formation of the oligonucleotide-vector complex).

Firstly, the penetration of the oligonucleotide into the THP-1 cells is verified using an oligonucleotide 21T coupled to a fluorescent label (called 21T*). The results show that the transfection brings about accumulation of the oligonucleotide in the cells.

The study of the inhibition of transcription of the TBFα gene was carried out using various concentrations of the oligonucleotide 21T. These concentrations are between 0.01 and 100 nM. FIG. 4 shows the effect of these various concentrations on the stimulated TNFα production. The PEI has no effect on the production of TNFα. Stimulation of the THP-1 cells with the combination LPS-IFN causes a significant increase in the production of TNFα. This increase is significantly inhibited by the oligonucleotide at the concentrations of 0.1 nM; 1 nM; 10 nM and 100 nM.

TNFα mRNA expression was studied by reverse transcription and then polymerase chain amplification. As shown in FIG. 5, treatment of the THP-1 cells with the combination LPS-IFN causes an increase in TNFα mRNAs, without affecting the expression of the gene encoding G3PDH. On the other hand, the increase in expression of the TNFα gene induced by LPS-IFN is reduced in a dose-dependant manner by the oligonucleotide 21T at the concentrations of 1 nM, 10 nM and 100 nM. The oligonucleotide 21T has no effect on the expression of the gene encoding G3PDH.

In order to determine the specificity of the action of the oligonucleotide on the stimulated production of TNFα in the THP-1 cells, a control oligonucleotide (21-mer with a composition identical to 21T, but with a different sequence) was used at the same concentrations as 21T, without any significant effect on the stimulated production of TNFα in the THP-1 cells. In addition, the 21T and control oligonucleotides at the concentrations used have no significant cell toxicity, as was determined using the method of lactate dehydrogenase release into the cell supernatant.

### Bibliographical References

(1) Elliot M J, Maini R N, Feldman M, Kalden J R, Antoni C, Smolen J S, Leeb B, Breedveld F C, Macfarlane J D, Bijil H, Woody J N. Randomised double-blind comparison of chimeric monoclonal antibody to tumour necrosis factor-α (CAC2) versus placebo in rheumatoid arthritis. Lancet 344, 1105-1110, 1994.

(2) Huizinga T W J, Binkman B M N, Verweij C L. Regulation of tumor necrosis factor-α production: basic aspects and pharmacological modulation. J Rheumatol 23, 416-419, 1996.

(3) Hartmann G, Krug A, Meoller J, Murphy J, Albrecht R, Endres S. Specific suppression of human tumor necrosis factor-alpha synthesis by antisense oligonucleotides. Antisense Nucleic Acid Drug Dev 6, 291-299, 1996.

(4) Braham A K, Smets P, Zalisz P, Sequence d'oligonucleotides anti-sens, anti-ARN messager du TNF alpha, procédé de préparation, application à titre de médicaments et compositions pharmaceutiques. [Sequence of anti-TNF-alpha messenger RNA antisense oligonucleotides, method of preparation, use as medicinal products and pharmaceutical compositions]. European patent EP 0 414 607 B1, 1995.

(5) Aggarwal B B, Rando R F, Hogan M E. Uses of triplex forming oligonucleotides for treatment of human diseases United States Patent, U.S. Pat. No. 5,650,316, 1997.

(6) Nedwin G K, Naylor S L, Sakaguchi A Y, Smith D, Jarrett-Nedwin J, Pennica D, Goeddel D V, Gray P W. Human lymphotoxin and tumor necrosis factor genes: structure, homology and chromosomal localization. Nucleic Acids Res 13, 6361-6373, 1985.

(7) Iraqi F, Teale A, Cloning and sequencing of TNFα genes of three inbred mouse strains, Immunogenetics 45 (6), 459-461, 1997.

(8) Tsuchiya S, Yamabe M, Yamaguchi Y, Kobayayashi Y, Konno T, Tada K. Establishment and characterization of a human acute monocytic leukemia cell line (THP-1). Int J Cancer, 26, 171-176, 1980.

(9) Eperon S, Jungi T W. The use of human monocytoid lines as indicators of endotoxin. J Immunol Methods, 194, 121-129, 1996.

(10) Chomczynski P, Sacchi N. Single-step method of RNA isolation by acid guanidinium thiocyanate-phenol-chloroform extraction. Anal Biochem, 162, 156-159, 1987.

(11) Abid A, Sabalovic N, Magdalou J. Expression and inducibility of UDP-glucuronosyltransferases 1-naphthol in human cultured hepatocytes and heptocarcinoma cells lines. Life Sci, 60, 1943-1951, 1997.

(12) Pennica D, Nedwin G E, Hayflick J S, Seeburg P H, Derynck R, Palladino M A, Kohr W J, Agarwal B B Gooddel D V. Human tumour necrosis factor: precursor structure, expression and homology to lymphotoxin. Nature, 312, 724-729, 1984.

(13) Arcari P, Martinelli R, Salvatore F. The complete sequence of full length cDNA for human liver glyceraldhyde-3-phosphate dehydrogenase: evidence for multiple mRNA species. Nucleic Acids Res, 12 9179-9189, 1984.

(14) Feng L, Xia Y, Garcia GE, Hwang D, Wilson CB, Involvement of reactive oxygen intermediates in cyclooxygenase-2 expression induced by interleukin-1, tumor necrosis factor-α, and lipopolysaccharide. J Clin Invest, 95, 1669-1675, 1995.

(15) Boussif O, Lezoualc'h F, Zanta M A, Mergny M D, Schermant D, Demeneix B, Behr J-P. A versatile vector for gene and oligonucleotide transfer into cells in culture and in vivo: Polyethylenimine. Proc Natl Acad Sci USA, 92, 7297-7301, 1995.

(16) Fang X, Li J J, Tan W. Using molecular beacons to probe molecular interactions between lactate dehydrogenase and single-stranded DNA. Anal Biochem, 72, 3280-3285, 2000.

(17) Bulyk M L, Gentalen E, Lockhart D J, Church G M. Quantifying DNA-protein interactions by double-stranded DNA arrays. Nat Biotechnol, 17, 573-577, 1999.

1. An oligonucleotide capable of directly or indirectly modulating the expression of the gene encoding tumor necrosis factor-alpha (TNFα) in various species and/or of genes under its control, characterized in that it comprises:
a constant portion-GGGGNGGG-, where N=T, A or U,
in the 5′ to 3′ or 3′ to 5′ orientation,
complementary to a target sequence,
two variable portions,
located on either side of the constant portion, conferring on the oligonucleotides a specificity of direct or indirect interaction with the target in the TNFα gene of the species considered,
in which the base at each of the ends, adjacent to the constant portion, is a purine.

2. The oligonucleotide as claimed in claim 1, characterized in that it comprises from 8 to 25 nucleotides, preferably of the order of 20-nucleotides.

3. The oligonucleotide as claimed in claimed 1 or 2, characterized in that the target consists of a deoxyribonucleic or ribonucleic acid sequence, or a protein which interacts directly or indirectly with this sequence.

4. The oligonucleotide as claimed in claim 3, characterized in that the target consists of a double helix.

5. The oligonucleotide as claimed in any one of the preceding claims, characterized in that the target is located upstream of the TATA box.

6. The oligonucleotide as claimed in any one of the preceding claims, characterized in that, in the Hominidae, the variable portions correspond to the sequences AAGAAA and AGGAGAG and are located, respectively, on the 5′ side and on the 3′ side in the 5′γ3′ direction, or on the 3′ side and on the 5′ side in the 3′φ5′ direction, in the Muridae, the variable portions correspond to the sequences TCGAAAA and AGAAGG, located, respectively, on the 5′ side and on the 3′ side in the 5′γ3′ direction, or on the 3′ side and on the 5′ side in the 3′φ5′ direction, and in the Bovidae, the variable portions correspond to the sequences CGGAAA and AGAAGTC, located, respectively, on the 5′ side and on the 3′ side in the 5′γ3′ direction, or on the 3′ side and on the 5′ side in the 3′φ5′ direction.

7. The oligonucleotide as claimed in any one of the preceding claims, characterized in that it is modified in its structure, in its sugars and/or in its bases, and in that it bears reactive groups, in particular at its variable ends.

8. A pharmaceutical composition, characterized in that it contains a therapeutically effective amount of at least one oligonucleotide as claimed in any one of claims 1 to 7, in combination with a pharmaceutically acceptable vehicle.

9. The pharmaceutical composition as claimed in claim 8, for treating conditions associated with an increased production TNFα, such as, for example, rheumatoid arthritis, Crohn's disease, septic shock, forms of inflammatory rheumatism, or sarcoidosis.

10. A kit comprising at least one oligonucleotide as claimed in any one of claims 1 to 7, and also the reagents for carrying out research or diagnosis.

11. The kit as claimed in claim 10, characterized in that the oligonucleotide(s) is (are) in solution or is (are) attached to a support, single-stranded or double-stranded, where appropriate coupled to a tracer.
